(19) United States
(12) Patent Application Publication
(10) Pub. No.: US 2011/0306848 A1
(43) Pub. Date: Dec. 15, 2011

(54) WOUND HEALING CHAMBER

(76) Inventors: Holger ENGEL, Taipei City (TW); Ming-Huei Cheng, Taipei City (TW); Eric Brey, Taipei City (TW); Emre Gazyakan, Taipei City (TW)

(21) Appl. No.: 13/160,592

(22) Filed: Jun. 15, 2011

(30) Foreign Application Priority Data
Jun. 15, 2010 (TW) 099119536

(51) Int. Cl. A61B 5/00

(52) U.S. Cl. 600/301

(57) ABSTRACT

A wound healing chamber is disclosed, which includes: a fixing plate with a first opening, wherein suturing holes are circularly formed around a peripheral section of the first opening, and the fixing plate is fixed on an object through the suturing holes; and a detachable cap mounted on the fixing plate, wherein the cap corresponds to the first opening, and the first opening is open or closed by the detachable cap.

### BACKGROUND OF THE INVENTION

1. Field of the Invention

The present invention relates to a wound healing chamber and, more particularly, to a wound healing chamber for observing the wound healing process in each of its different phases in animal tests.

2. Description of Related Art

The healing process of wounds is very complicated, and several different cells, tissue, and proteins are involved therein. In general, the wound healing process can be divided into three phases including inflammation, proliferation, and remodeling. The mechanism of the inflammatory phase comprises granulation, wound contraction, and epithelialization. Proliferation and remodeling are necessary to create a biomechanical stable wound.

To study the wound healing process in each of its phase or the influence of drugs on the wound regions, researchers cut skin or remove partial tissue parts of an object such as a mouse, and then apply a wound observation ring 1 on the object, as shown in FIG. 1. The wound observation ring 1 has an opening 11, and the wound of the mouse is exposed from the opening 11. Hence, researchers can observe the status of the wound healing on the mouse from the opening 11 of the wound observation ring 1.

However, the traditional wound observation ring 1 is fixed on the mouse only through an adhesive. When the mouse struggles or scratches its skin, the wound observation ring 1 may be torn off. Therefore, the observation may be influenced.

In addition, the traditional wound observation ring 1 is not installed with a cap. In this case, foreign matters may drop into the wound region during the process of wound healing. In addition, the wound region is exposed to the air, so the danger of bacteria infection is increased.

Therefore, in order to obviate the aforementioned problem, it is desirable to provide a wound healing device, which can be used for observing the mechanism of the wound healing, and the influence of drugs on the wound regions.

### SUMMARY OF THE INVENTION

The object of the present invention is able to provide a wound healing chamber to investigate the wound healing process in detail in each of its different phases. It is helpful to study the mechanism of wound healing, and the influence of drugs or other topical agents on the wound regions in a controlled and modifiable setting and area. Furthermore, it can prevent foreign matters dropping into the wound regions and decreasing the possibility of bacteria infection.

To achieve the object, the wound healing chamber of the present invention comprises: a fixing plate with a first opening, wherein plural suturing holes are formed around a peripheral section of the first opening, and the fixing plate is fixed on an object through the suturing holes; and a detachable cap mounted on the fixing plate, wherein the cap corresponds to the first opening, and the first opening is open or closed by the detachable cap. More specifically, the fixing plate of the wound healing chamber of the present invention is a flange.

The wound healing chamber of the present invention can be fixed on the object through sutures and/or glue, so the wound healing chamber is not torn off from the object when the object struggles or scratches its skin. In addition, the wound healing chamber of the present invention comprises a detachable cap, which can prevent foreign matters dropping into the wound region to decrease the possibility of bacteria infection.

Hence, the wound healing chamber of the present invention can facilitate the researchers to study the mechanism of wound healing. Furthermore, the wound healing chamber of the present invention also can facilitate the researchers to observe the influence of various drugs and other topical agents in a controlled and modifiable area/chamber on the healing of wound regions.

According to the wound healing chamber of the present invention, the number of the suturing holes circularly formed around the peripheral section of the first opening is 6 to 10, preferably.

In addition, the wound healing chamber of the present invention may further comprise a pivoting unit to mount the detachable cap on the fixing plate. Alternatively, the detachable cap can screw on the fixing plate.

According to the wound healing chamber of the present invention, a protrusion is disposed on an inner edge of the first opening, and plural first fixing holes are disposed on the protrusion.

Hence, the edge of the wound can be fixed when the sutures pass through the first fixing holes. When the edge of the wound is fixed, wound contraction can be inhibited, and therefore the phases of granulation and epithelialization can be observed.

Furthermore, according to the wound healing chamber of the present invention, the gaps between adjacent suturing holes are not particularly limited. Preferably, the gaps between adjacent suturing holes are the same. In addition, the gaps between adjacent first fixing holes are also not particularly limited. Preferably, the gaps between adjacent first fixing holes are the same.

According to the wound healing chamber of the present invention, the detachable may have an observation window, which corresponds to the first opening of the fixing plate. Hence, the researchers can observe the status of the wound region without opening the detachable cap.

According to one aspect of the present invention, the wound healing chamber may further comprise a drug delivery unit, which is mounted in the observation window. Preferably, the material of the drug delivery unit is silicon-derived material, such as silica gel. When drugs or agents are injected into the drug delivery unit, the drugs or agents can be delivered to the wound region.

Hence, the researchers can administer drugs or topical agents into the wound region without opening the detachable cap, to observe the influence of drugs on the wound region.

According to another aspect of the present invention, the wound healing chamber may further comprise a transparent observation unit, which is mounted in the observation window. The transparent observation unit can be any transparent material such as a glass plate or a plastic plate, even a lens to enlarge the wound region. In addition, a scale may be formed on the transparent observation unit. Hence, the diameter or width of the wound region can be measured by the scale on the transparent observation unit, and therefore the healing rate of the wound region can be observed.

In addition, according to further aspect of the present invention, the wound healing chamber may further comprise plural first pins disposed on a bottom surface of the detachable cap. Each first pins respectively corresponds to one first fixing hole, and the first pins shield into the first fixing holes when the first opening is closed by the detachable cap.

Hence, the edge of the wound region can be fixed through the first pins without using sutures, so the wound contraction can be inhibited. Therefore, the step of fixing the edge of the wound region through sutures can be omitted, and the time for mounting the wound healing chamber on the object can be saved.

Furthermore, according to further another aspect of the present invention, the wound healing chamber may further comprise a bottom plate, which has a second opening corresponding to the first opening of the fixing plate. The skin of the object is disposed between the bottom plate and the fixing plate, so the wound contraction and the epithelialization can be inhibited. Therefore, the granulation of the wound region can be observed solely. In addition, according to the wound healing chamber of the present aspect, plural second pins may be disposed on a bottom surface of the protrusion, plural third pins may be disposed on an upper surface of the bottom plate, plural second fixing holes are formed on the bottom plate, the second pins correspond to the second fixing holes to shield into the second fixing holes, and the third pins correspond to the first fixing holes to shield into the first fixing holes. When the second pins shield into the second fixing holes and the third pins shield into the first fixing holes, the relative position between the fixing plate and the bottom plate can be maintained. Furthermore, the first fixing holes and the second pins are alternately disposed.

In addition, according to further another aspect of the present aspect, a skin support notch is disposed on an inner edge of the first opening, wherein the skin support notch comprises a side wall connecting to the fixing plate, and the side wall is formed with plural third fixing holes. When the edge of the wound is fixed with the skin support notch, both the phases of wound contraction and epithelialization can be inhibited. Hence, the phases of granulation can be observed. In addition, the wound healing chamber of the present aspect may further comprise plural first pins disposed on a bottom surface of the detachable cap, wherein each third fixing hole of the skin support notch corresponds to one first pin respectively. Hence, the first pins can shield into the third fixing holes when the first opening is closed by the detachable cap.

According to all aspects of the present invention, the material of the wound healing chamber can be any biocompatible material, which can be selected from the group consisting of titanium, stainless steel, silicon-derived material such as silica gel, acrylic, and a combination thereof. In addition, the wound healing chamber of the present invention is used in animal tests.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a wound observation ring mounted on a rat;

FIG. 2 is a perspective view of a wound healing chamber of Embodiment 1 of the present invention;

FIG. 3 is a perspective view of a wound healing chamber of Embodiment 1 of the present invention, which is mounted on a mouse;

FIG. 4 is a perspective view of a wound healing chamber of Embodiment 2 of the present invention;

FIG. 5 is a perspective view of a wound healing chamber of Embodiment 3 of the present invention;

FIG. 6 is a perspective view of a wound healing chamber of Embodiment 4 of the present invention;

FIG. 7 is a cross-sectional view of a wound healing chamber of Embodiment 4 of the present invention, which is mounted on an animal;

FIG. 8 is a cross-sectional view of a wound healing chamber of Embodiment 5 of the present invention, which is mounted on an animal;

FIG. 9 is a perspective view of a wound healing chamber of Embodiment 6 of the present invention; and

FIG. 10 is a cross-sectional view of a wound healing chamber of Embodiment 6 of the present invention, which is mounted on an animal.

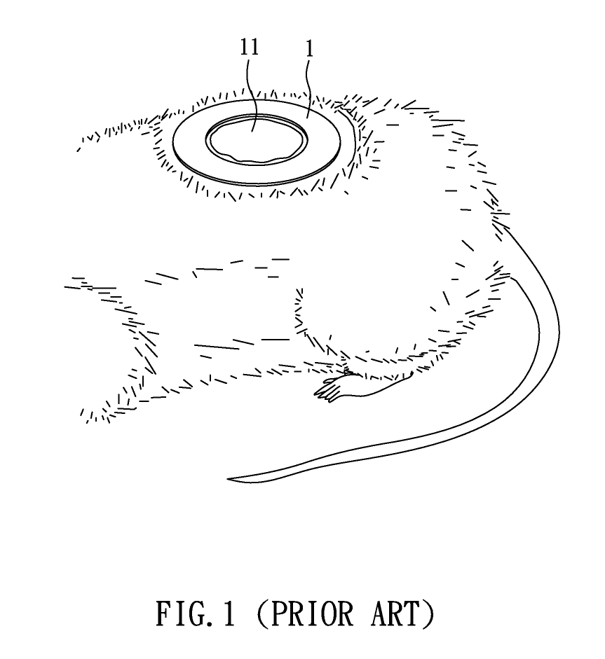

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Embodiment 1

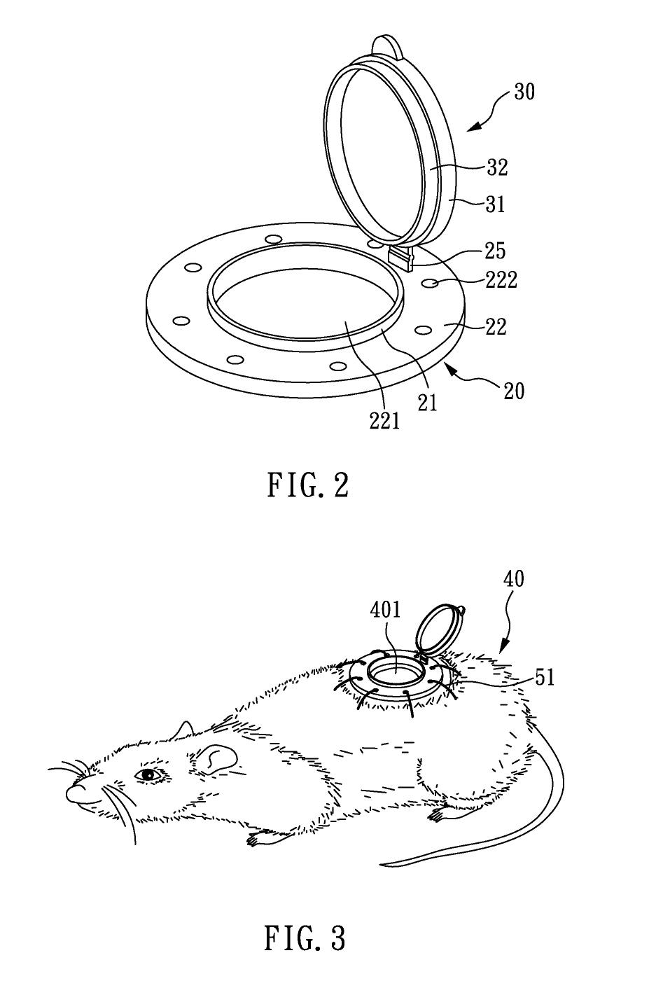

FIG. 2 is a perspective view of a wound healing chamber of the present embodiment, and FIG. 3 is a perspective view of the wound healing chamber of the present embodiment mounted on a rat.

As shown in FIG. 2 and FIG. 3, the wound healing chamber of the present embodiment comprises: a base 20, and a detachable cap 30 mounted on the base 20. The base 20 comprises a first tube 21, and a fixing plate 22 extended from the edge of the first tube 21. The detachable cap 30 comprises an upper cap 31 and a second tube 32, and the upper cap 31 is disposed on the outer edge of the second tube 32. Herein, the outer diameter of the second tube 32 is slightly smaller than the inner diameter of the first tube 21. Hence, when the detachable cap 30 is mounted on the base 20, the second tube 32 can installed into the first tube 21 to form a chamber. In the present embodiment, the detachable cap 30 is fixed on the fixing plate 22 through a pivoting unit 25.

In addition, a first opening 221 is disposed on the fixing plate 22, and plural suturing holes 222 are circularly formed around a peripheral section of the first opening 221. The gaps between two adjacent suturing holes 222 are the same. When the wound healing chamber of the present embodiment is disposed on an object 40, sutures 51 pass through the suturing holes 222 to fix the fixing plate 22 on the skin of the object 40, and only the wound region 401 of the object 40 is exposed from the first opening 221, as shown in FIG. 3.

The detachable cap 30 corresponds to the first opening 221. Hence, when the detachable cap 30 is detached, the wound region 401 is exposed from the first opening 221, and the observation or the treatment of the wound region 401 can be performed. When the detachable cap 30 is mounted, the first opening 221 is closed, and the dropping of foreign matters or the bacteria infection can be prevented.

In addition, the wound healing chamber of the present embodiment is used on animal tests, so the material of the wound healing chamber have to be a biocompatible material, such as titanium, stainless steel, silicon derived material such as silica gel, and acrylic. In the present embodiment, the material of the wound healing chamber is titanium.

### Embodiment 2

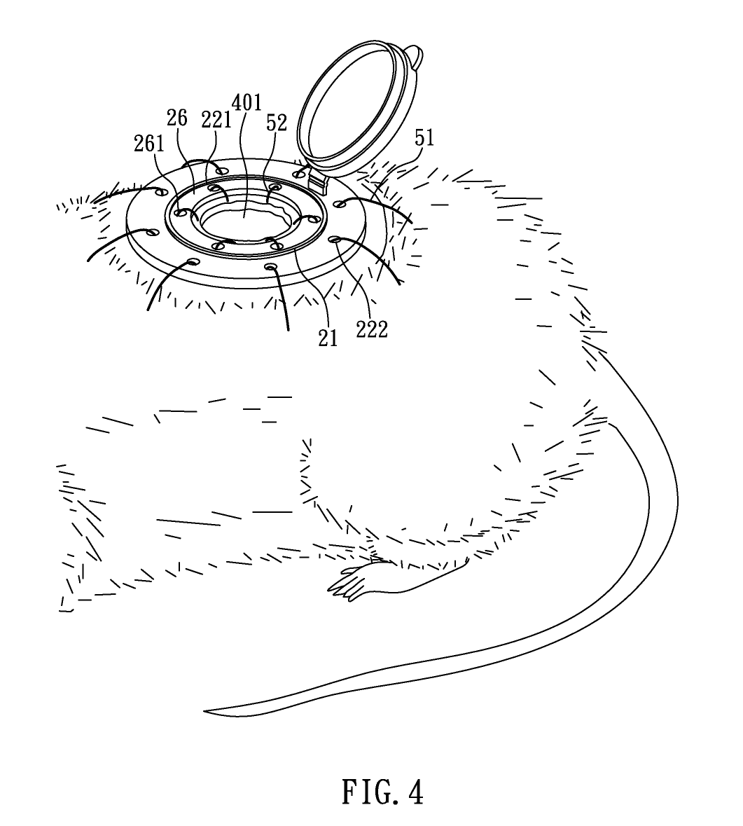

FIG. 4 is a perspective view of the wound healing chamber of the present embodiment mounted on an animal.

As shown in FIG. 4, the structure of the wound healing chamber of the present embodiment is similar to that of Embodiment 1, except that a protrusion 26 is disposed on an inner edge of the first opening 221, i.e. a protrusion 26 is disposed on an inner edge of the first tube 21. Herein, plural first fixing holes 261 are circularly formed on the protrusion 26, and the gaps between two adjacent first fixing holes 261 are the same. When sutures 52 pass through the first fixing holes 261 to fix the edge of the wound region 401, the wound contraction of the wound region can be inhibited. Therefore, the granulation and the epithelialization of the wound region 401 can be observed.

### Embodiment 3

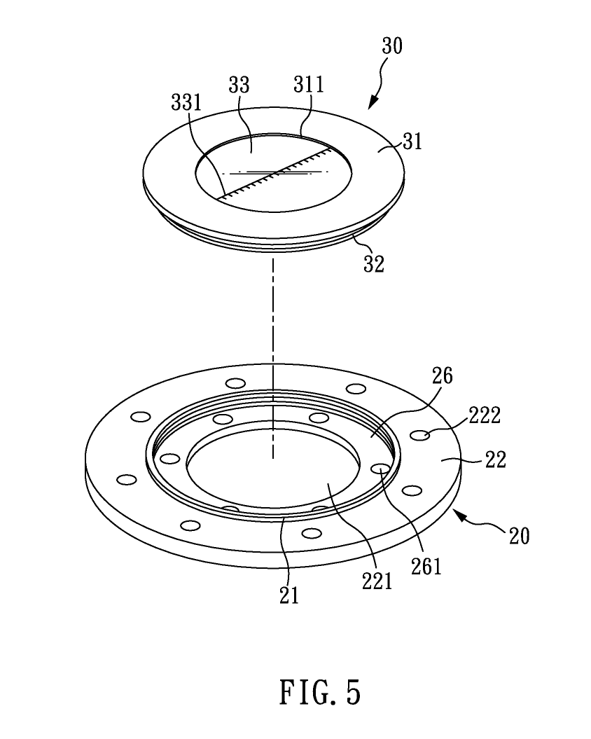

FIG. 5 is a perspective view of the wound healing chamber of the present embodiment.

As shown in FIG. 5, the structure of the wound healing chamber of the present embodiment is similar to that of Embodiment 2, except that the detachable cap 30 of the present embodiment crews on the base 20. More specifically, according to the wound healing chamber of the present invention, the outer side of the second tube 32 of the detachable 30 has outer threads, and the inner side of the first tube 21 of the base 20 has inner threads. When the outer threads and the inner threads are screwed together, the detachable cap 30 can be mounted on the base 20. When the outer threads and the inner threads are departed from each other, the detachable cap 30 can be detached from the base 20.

In addition, according to the wound healing chamber of the present embodiment, the upper cap 31 of the detachable cap 30 is formed with an observation window 311, and the observation window 311 corresponds to the first opening 221 of the fixing plate 22. Therefore, the changes of the wound region and/or the healing process can be observed through the observation window 311 without detaching the detachable cap 30 from the base 20.

In addition, the wound healing chamber of the present embodiment further comprises a transparent observation unit 33, which is mounted in the observation window 311. In the present embodiment, the transparent observation unit 33 is a glass plate, and a scale 331 is formed on the transparent observation unit 33. Hence, the diameter or the width of the wound region can be measured through the scale, and therefore the healing rate of the wound region can be observed.

### Embodiment 4

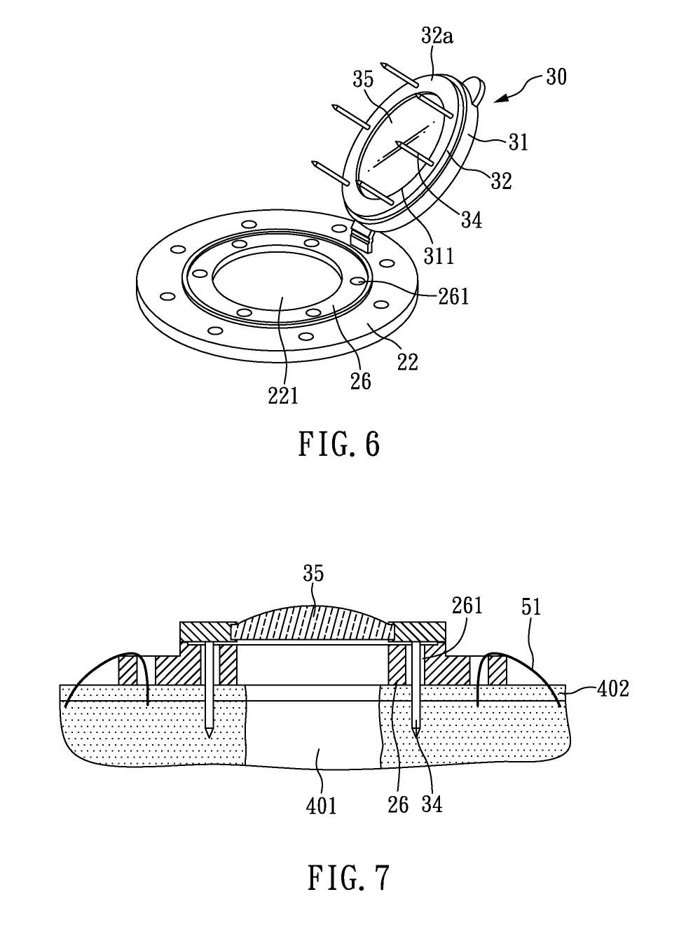

FIG. 6 is a perspective view of a wound healing chamber of the present embodiment, and FIG. 7 is a cross-sectional view of the wound healing chamber of the present embodiment mounted on an animal.

As shown in FIG. 6, the structure of the wound healing chamber of the present embodiment is similar to that of Embodiment 2, except that the wound healing chamber of the present embodiment further comprises plural first pins 34 disposed on a bottom surface 32a of the second tube 32 of the detachable cap 30. In addition, each first pins 34 corresponds to one first fixing hole 261 of the protrusion 26. When the detachable 30 is mounted to close the first opening 221 of the fixing plate 22, the first pins 34 shield into the first fixing holes 221, as shown in FIG. 7. Hence, according to the wound healing chamber of the present embodiment, the edge of the wound region 401 is fixed with the first pins 34, and without using any sutures. Therefore, the time for fixing the edge of the wound region can be saved.

In addition, according to the wound healing chamber of the present embodiment, the upper cap 31 of the detachable cap 30 is formed with an observation window 311, and the observation window 311 corresponds to the first opening 221 of the fixing plate 22. Furthermore, a drug delivery unit 35 is mounted in the observation window 311. Hence, it is possible to deliver drugs without opening the detachable cap 30. In the present embodiment, the material of the drug delivery unit 35 is silicon-derived material, such as silica gel.

### Embodiment 5

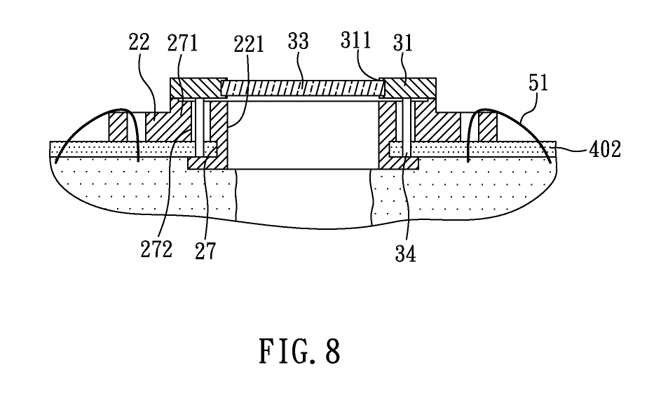

FIG. 8 is a cross-sectional view of the wound healing chamber of the present embodiment mounted on an animal.

As shown in FIG. 8, the structure of the wound healing chamber of the present embodiment is similar to that of Embodiment 1, except that a skin support notch 27 is disposed on an inner edge of the first opening 221, and a side wall 271 of the skin support notch 27, which connect to the fixing plate 22, is formed with plural third fixing holes 272. In addition, the wound healing chamber of the present embodiment further comprises plural first pins 34 disposed on a bottom surface of the upper cap 31, and the third fixing holes 272 of the skin support notch 27 correspond to the first pins 34. Hence, when the detachable cap 31 is mounted on the fixing plate 22 to close the first opening 221, the first pins 34 can shield into the third fixing hole 272. Therefore, according to the wound healing chamber of the present embodiment, the tissue 402 around the wound region can be fixed with the first pins 34, so the time for fixing the edge of the wound region can be saved. Furthermore, the wound contraction and epithelialization is inhibited to investigate the process of granulation solely.

In addition, according to the wound healing chamber of the present embodiment, the upper cap 31 of the detachable cap 30 is formed with an observation window 311, and the observation window 311 corresponds to the first opening 221 of the fixing plate 22. In the present embodiment, a transparent observation unit 33, which is a lens, is mounted in the observation window 311.

### Embodiment 6

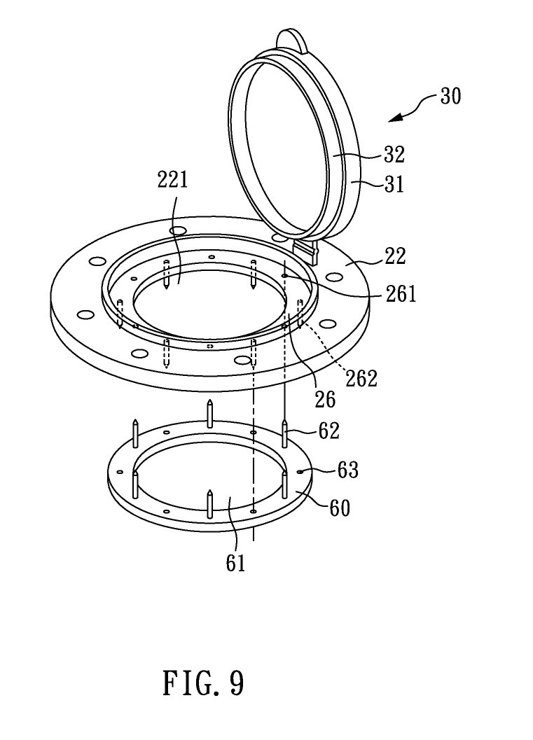

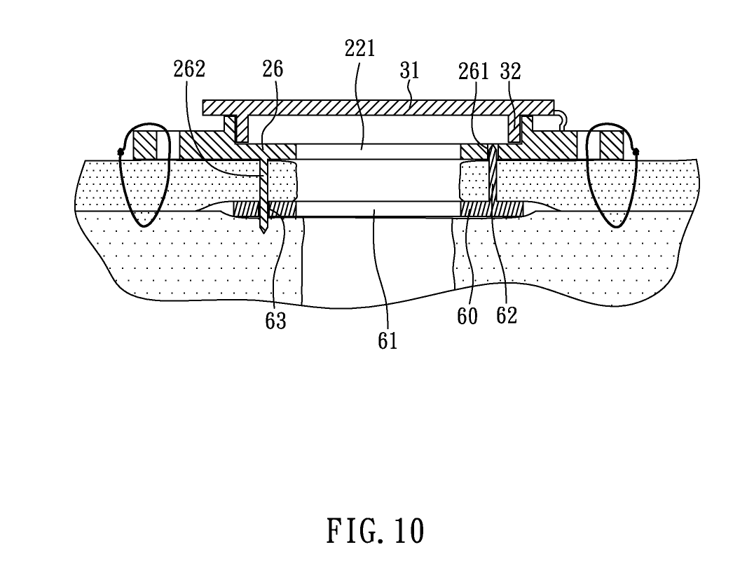

FIG. 9 is a perspective view of a wound healing chamber of the present embodiment, and FIG. 10 is a cross-sectional view of the wound healing chamber of the present embodiment mounted on an animal.

As shown in FIG. 9, the structure of the wound healing chamber of the present embodiment is similar to that of Embodiment 1, except that the plural second pins 262 are disposed on a bottom surface of a protrusion 26, and the wound healing chamber of the present embodiment further comprises a bottom plate.

As shown in FIG. 9 and FIG. 10, according to the wound healing chamber of the present embodiment, the bottom plate 60 has a second opening 61, which corresponds to the first opening 221 of the fixing plate 22. In addition, plural third pins 62 are disposed on an upper surface of the bottom plate 60, and the plural second fixing holes 63 are formed in the bottom plate 60. In addition, the first fixing holes 261 of the protrusion 26 and the second pins 262 are alternately disposed, and the second fixing holes 63 of the bottom plate 60 and the third pins 62 are alternately disposed.

Each second pins 262 of the protrusion 26 respectively corresponds to one second fixing hole 63 of the bottom plate 60, and each third pins 62 of the bottom plate 60 respectively corresponds to one fixing hole 261 of the protrusion 26. Hence, when the bottom plate 60 is mounted with the fixing plate 22, skin of an object can be fixed between the bottom plate 60 and the fixing plate 22. In addition, the second pins 262 can shield into the second fixing holes 63, and the third pins 62 can shield into the first fixing holes 261. Therefore, the wound contraction of the skin can be inhibited, so the phases of epithelialization and granulation can be observed.

In conclusion, the present invention provides a wound healing chamber, which can be fixed on an object through sutures or pins to prevent the wound healing chamber torn off. In addition, the detachable cap of the wound healing chamber of the present invention can prevent foreign matters dropping into the wound region, to decrease the possibility of bacteria infection. Furthermore, the protrusion, the skin support notch, and/or the bottom plate with pins can also be used to observe the mechanism of wound healing in each of its different phase of wound healing

Although the present invention has been explained in relation to its preferred embodiment, it is to be understood that many other possible modifications and variations can be made without departing from the spirit and scope of the invention as hereinafter claimed.

What is claimed is:

1. A wound healing chamber, comprising:
a fixing plate with a first opening, wherein plural suturing holes are formed around a peripheral section of the first opening, and the fixing plate is fixed on an object through the suturing holes; and
a detachable cap mounted on the fixing plate, wherein the cap corresponds to the first opening, and the first opening is open or closed by the detachable cap.

2. The wound healing chamber as claimed in claim 1, further comprising a pivoting unit to mount the detachable cap on the fixing plate.

3. The wound healing chamber as claimed in claim 1, wherein the detachable cap screws on the fixing plate.

4. The wound healing chamber as claimed in claim 1, wherein a protrusion is disposed on an inner edge of the first opening, and plural first fixing holes are disposed on the protrusion.

5. The wound healing chamber as claimed in claim 1, wherein the detachable has an observation window, which corresponds to the first opening of the fixing plate.

6. The wound healing chamber as claimed in claim 5, further comprising a drug delivery unit, which is mounted in the observation window.

7. The wound healing chamber as claimed in claim 6, wherein the material of the drug delivery unit is silicon derived material.

8. The wound healing chamber as claimed in claim 5, further comprising a transparent observation unit, which is mounted in the observation window.

9. The wound healing chamber as claimed in claim 8, wherein the transparent observation unit is a glass plate, a plastic plate, or a lens.

10. The wound healing chamber as claimed in claim 8, wherein a scale is formed on the transparent observation unit.

11. The wound healing chamber as claimed in claim 1, wherein the gaps between adjacent suturing holes are the same.

12. The wound healing chamber as claimed in claim 4, wherein the gaps between adjacent first fixing holes are the same.

13. The wound healing chamber as claimed in claim 4, further comprising plural first pins disposed on a bottom surface of the detachable cap, wherein the first pins correspond to the first fixing holes, and the first pins shield into the first fixing holes when the first opening is closed by the detachable cap.

14. The wound healing chamber as claimed in claim 4, further comprising a bottom plate, wherein the bottom plate has a second opening, and plural second fixing holes, the second opening corresponds to the first opening of the fixing plate, plural second pins are disposed on a bottom surface of the protrusion, plural third pins are disposed on an upper surface of the bottom plate, the second pins correspond to the second fixing holes to shield into the second fixing holes, and the third pins correspond to the first fixing holes to shield into the first fixing holes.

15. The wound healing chamber as claimed in claim 15, wherein the first fixing holes and the second pins are alternately disposed.

16. The wound healing chamber as claimed in claim 1, wherein a skin support notch is disposed on an inner edge of the first opening, the skin support notch comprises a side wall connecting to the fixing plate, and the side wall is formed with plural third fixing holes.

17. The wound healing chamber as claimed in claim 16, further comprising plural first pins disposed on a bottom surface of the detachable cap, wherein the third fixing holes of the skin support notch correspond to the first pins, and the first pins shield into the third fixing holes when the first opening is closed by the detachable cap.

18. The wound healing chamber as claimed in claim 1, wherein the material of the wound healing chamber is selected from the group consisting of titanium, stainless steel, silicon derived material, acrylic, and a combination thereof.
